(19) United States
(12) Patent Application Publication
(10) Pub. No.: US 2025/0303122 A1
(43) Pub. Date: Oct. 2, 2025

(54) SYSTEMS AND METHODS FOR INSULATIVE TEMPERATURE CONTROL IN DELIVERY OF THERMAL LIQUID TREATMENT

(71) Applicant: Intuitive Surgical Operations, Inc., Sunnyvale, CA (US)

(72) Inventors: Erik M. Henne, Mercer Island, WA (US); Phoenix M. Abernathy, Woodinville, WA (US); Robert L. Barry, Seattle, WA (US); Joseph D. Bogusky, San Jose, CA (US); Jacqueline L. Corso, Bothell, WA (US); Cassandra R. Saleira, Bothell, WA (US); Alexander M. Vanden Bussche, Seattle, WA (US); Jesse L. Via, Renton, WA (US)

(21) Appl. No.: 19/097,673

(22) Filed: Apr. 1, 2025

Related U.S. Application Data
(60) Provisional application No. 63/573,205, filed on Apr. 2, 2024.

(51) Int. Cl. A61M 25/10; A61M 25/00

(52) U.S. Cl. CPC A61M 25/10; A61M 25/005; A61M 2202/0007; A61M 2202/04; A61M 2205/0216

(57) ABSTRACT

A flexible elongate device comprises a wall defining a lumen and a liquid delivery channel extending within the lumen. The liquid delivery channel is configured to convey a treatment liquid. The flexible elongate device also comprises an insulation chamber extending along the liquid delivery channel within the lumen. The insulation chamber contains a static insulator. The flexible elongate device also comprises a support structure that constrains an axial position of the liquid delivery channel with respect to the lumen.

### CROSS-REFERENCED APPLICATIONS

This application claims priority to and benefit of U.S. Provisional Application No. 63/573,205 filed Apr. 2, 2024 and entitled “Systems and Methods for Insulative Temperature Control in Delivery of Thermal Liquid Treatment,” which is incorporated by reference herein in its entirety

### FIELD

Examples described herein are related to systems and methods for endoluminal thermal treatment of diseased anatomy using insulating chambers to maintain a temperature of a treatment liquid.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. In some examples, a minimally invasive medical instrument may be a thermal energy treatment instrument for use within an endoluminal passageway of a patient anatomy.

### SUMMARY

The following presents a simplified summary of various examples described herein and is not intended to identify key or critical elements or to delineate the scope of the claims.

In some examples, a flexible elongate device comprises a wall defining a lumen and a liquid delivery channel extending within the lumen. The liquid delivery channel is configured to convey a treatment liquid. The flexible elongate device also comprises an insulation chamber extending along the liquid delivery channel within the lumen. The insulation chamber contains a static insulator. The flexible elongate device also comprises a support structure that constrains an axial position of the liquid delivery channel with respect to the lumen.

In some examples, a system comprises a delivery device including a working channel and a flexible elongate device configured to extend through the working channel. The flexible elongate device includes a liquid delivery channel. An insulation chamber extends along the liquid delivery channel. The insulation chamber contains a static insulator. The flexible elongate device also includes a support structure that constrains an axial position of the liquid delivery channel within the flexible elongate device.

In some examples, a method comprises providing a heated liquid to a liquid delivery channel of a flexible elongate device. The heated liquid within the liquid delivery channel is insulated by one or more insulation chambers of the flexible elongate device surrounding the liquid delivery channel. The insulation chamber contains a static insulator. The method also comprises delivering the heated liquid through a distal end of the liquid delivery channel.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a patient anatomy according to some examples.

FIG. 2 illustrates a cross-sectional view of the distal end portion of a medical instrument system, according to some examples.

FIG. 3 is a schematic illustration of a medical system, according to some examples.

FIG. 4 illustrates a flexible elongate device, according to some examples.

FIG. 5 illustrates a flexible elongate device, according to some examples.

FIG. 6 illustrates a flexible elongate device, according to some examples.

FIG. 7 illustrates a flexible elongate device, according to some examples.

FIG. 8 illustrates a flexible elongate device, according to some examples.

FIG. 9 illustrates a flexible elongate device, according to some examples.

FIG. 10 illustrates a flexible elongate device, according to some examples.

FIG. 11 illustrates a flexible elongate device, according to some examples.

FIG. 12 illustrates a flexible elongate device, according to some examples.

FIG. 13 illustrates a flexible elongate device, according to some examples.

FIG. 14 is a flowchart illustrating a method for applying a thermal energy treatment to an endoluminal passageway, according to some examples.

FIG. 15 illustrates an anatomic passage occlusion technique, according to some examples.

FIG. 16 illustrates an anatomic passage occlusion technique, according to some examples.

FIG. 17 illustrates an anatomic passage occlusion technique, according to some examples.

FIG. 18 is robot-assisted medical system, according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The technology described herein provides techniques and treatment systems for endoluminal thermal treatment of diseased tissue. Although the examples provided herein may refer to treatment of lung tissue and pulmonary disease, it is understood that the described technology may be used in treating artificially created lumens or any endoluminal passageway or cavity, including in a patient trachea, colon, intestines, stomach, liver, kidneys and kidney calices, brain, heart, circulatory system including vasculature, fistulas, and/or the like. In some examples, treatment described herein may be referred to as endobronchial thermal liquid treatment and may be used in procedures to treat lung tumors and/or chronic obstructive pulmonary disease (COPD) that may include one or more of a plurality of disease conditions including chronic bronchitis, emphysema, and bronchiectasis.

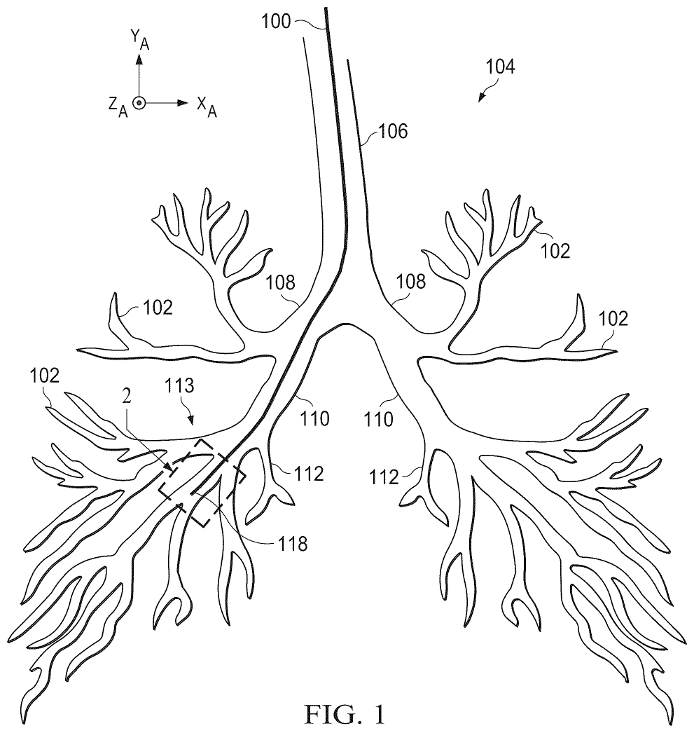

FIG. 1 illustrates a medical instrument system 100 extending within branched anatomic passageways or airways 102 of an anatomical structure 104. In some examples the anatomic structure 104 may be a lung and the passageways 102 that include the trachea 106, primary bronchi 108, secondary bronchi 110, and tertiary bronchi 112. The anatomic structure 104 has an anatomical frame of reference (X_{A}, Y_{A}, Z_{A}). A distal end portion 118 of the medical instrument 100 may be advanced into an anatomic opening (e.g. a patient mouth) and through the anatomic passageways 102 to perform a medical procedure, such as an endoluminal thermal energy treatment, at or near target tissue located in a region 113 of the anatomic structure 104.

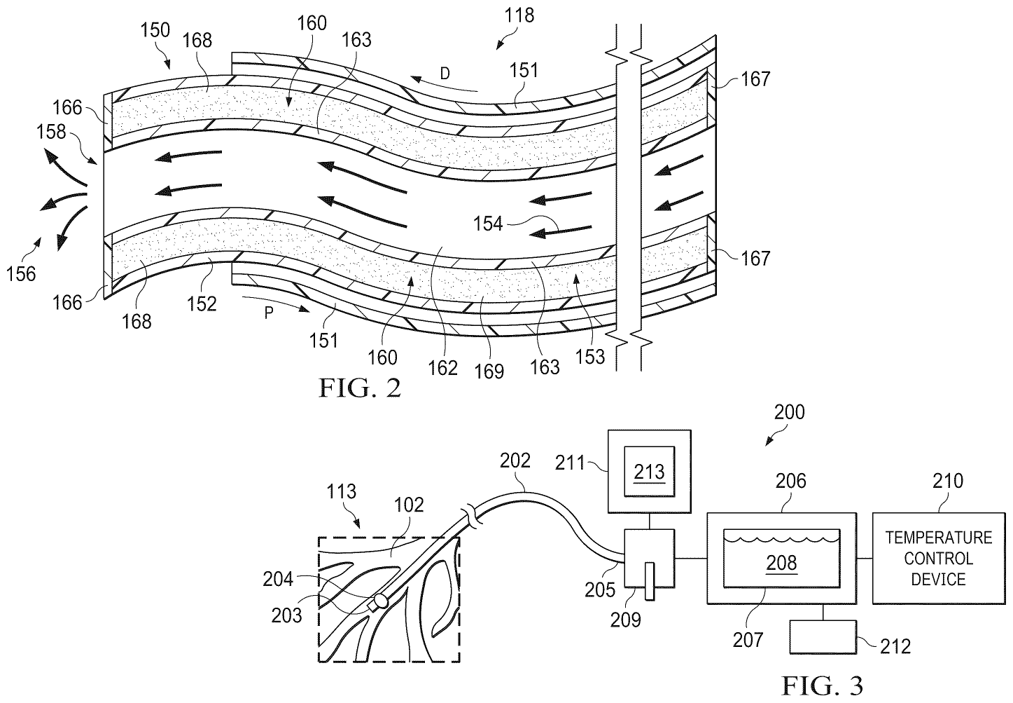

FIG. 2 illustrates the distal end portion 118 of the medical instrument system 100. The flexible elongate device 150 includes an outer wall 152 surrounding a lumen 153. An insulation chamber 160 and a liquid delivery channel 162 extend within the lumen 153 bounded by the outer wall 152. The liquid delivery channel 162 may have a channel wall 163 and may carry a heated treatment liquid 154 from a proximal end portion of the flexible elongate device 150, through the distal end portion 118, and may deliver the heated treatment liquid 154 to an area 156 distal of a distal opening 158 of the liquid delivery channel 162. The insulation chamber 160 may be bounded by a distal wall 166 and a proximal wall 167 to resist or prevent inflow or outflow of fluids or other materials. The insulation chamber 160 may contain a static insulator 168 that may be static in that it may not move or circulate into or out of the chamber 160 while the heated treatment liquid 154 flows through the liquid delivery channel 162. In some examples, the insulation chamber 160 may contain a static insulator 168 such as air or solid insulation material. The solid insulation material may be a continuous material such as a foam or may include particles such as synthetic down or fiberglass. In other examples, the static insulator may be a vacuum formed within the insulation chamber 160. A vacuum may include an absence of all substance such as air or may include a low density or low pressure air (as compared to ambient pressure). The insulation chamber 160 may insulate the liquid delivery channel 162 to maintain or control a temperature of the heated treatment liquid 154 along the length of the flexible elongate device 150. The insulation chamber 160 may reduce, minimize, or eliminate heat transfer between liquid delivery channel 162 and the outer wall 152. Optionally, the flexible elongate device 150 may extend through a working channel of a flexible elongate delivery device 151, such as a bronchoscope, endoscope, or a catheter. In some examples, the flexible elongate device 150 may be robotically-assisted. In some examples, the flexible elongate device 150 may be manually controlled. In some examples, the flexible elongated delivery device 151 may be robotically-assisted. In some examples, markers, fiducials, bands, or other visible indicators may be fixed to or incorporated into the flexible elongate device 150 (e.g., on the distal wall 166 or the outer wall 152. The visible indicators may be visible, for example with optical imaging (e.g., reflectance imaging, fluorescence imaging), to allow for improved visualization during the distal placement of the distal end of the flexible elongate device. In some examples, the visible indicators may be formed of nylon or PEBAX.

In some examples, during a treatment procedure using the medical instrument system 100, the treatment liquid 154 may be a heated saline or gel used to provide a thermal treatment to the region 113 of the anatomic structure. In these examples, the insulation chamber 160 helps maintain the temperature of the treatment liquid 154. In some examples, the treatment liquid 154 may enter the liquid delivery channel at a temperature of between approximately 50° C. and 99° C. and may be maintained at approximately the same temperature along the length of the flexible elongate device 150 by the insulation chamber 160. In some examples, the temperature of the treatment liquid entering the flexible elongate device may be between approximately 95° C. and 99° C. In some examples, for liquids that have vaporization temperatures greater than 99° C., the temperature of the treatment liquid may be greater than 99° C. For example, a treatment liquid at temperatures of approximately 100-115 C may be maintained in a liquid phase in a pressurized state. In some examples, the flow rate of the treatment liquid 154 may be approximately 70 ml/min. In other examples, the flow rate of the treatment liquid 154 may be between approximately 0.2 to 1.0 ml/sec.

In some examples, the insulation chamber 160 may be preheated before the heated treatment liquid 154 is flowed through the liquid delivery channel 162. For example, heated air may be pumped into the insulation chamber 160 before the heated treatment liquid is flowed through the liquid delivery channel 162. The heated air may heat the channel wall 163. The heated air may be sealed in the insulation chamber 160 or may be vacated prior to sealing a vacuum in the insulation chamber 160. In other examples, a heated liquid may be pumped into the insulation chamber to heat the channel wall 163. The heated liquid may be evacuated and the insulation chamber 160 dried before a vacuum or substance 169 is sealed in the insulation chamber.

FIG. 3 illustrates a medical instrument system 200. The medical instrument system 200 may be, for example, the medical instrument system 100 and may include a flexible elongate device 202 with an occlusion device 204 coupled to a distal end portion 203 of the flexible elongate device 202. The flexible elongate device 202 may be similar to flexible elongate device 150 and may, in some embodiments, be inserted through an outer sheath (e.g. delivery device 151). In some examples the flexible elongate device 202 and/or the sheath may be manually or robotically actuated or delivered using a robotically-assisted flexible elongate device. The occlusion device 204 may expand within a passageway 102 to prevent the flow of treatment liquid released from the distal end portion 203 of the flexible elongate device 202 proximally into the passageway 102. The occlusion device 204 may be, for example, an inflatable device such as a balloon fillable with an inflation medium such as air, saline, or another type of suitable fluid for expanding the balloon. The flexible elongate device 202 may be coupled to and in fluid communication with a fluid source 211 including a reservoir 213 that contains the inflation medium. In some examples, the proximal end portion 205 of the flexible elongate device 202 may be connected to the fluid source 211 via a control valve 209. In some embodiments the fluid source 211 may be a syringe including a fluid reservoir for containing a predetermined amount of inflation medium that may be injected into the occlusion device 204 to inflate the occlusion device. In some embodiments, for example, a 1 cm balloon occlusion device may be inflated with 1 cc of air from the syringe inflation device. In other examples, larger or smaller balloon occlusion devices and larger or smaller volumes of air may be used.

A proximal end portion 205 of the flexible elongate device 202 may be coupled to and in fluid communication with a fluid source 206 including a reservoir 207 that contains a non-compressible fluid 208, such as a liquid. In some examples, the proximal end portion 205 may be coupled to the fluid source 206 via the control valve 209 or a separate control valve. The temperature of the liquid 208 may be maintained by a temperature control device 210. The temperature control device 210 may include a heating system for heating the liquid 208. The heating system may include a heat generator, a temperature sensor, and other temperature regulation and generation components. In some examples, the heating system may heat the liquid 208 in the reservoir 207 with resistive heating, radiofrequency heating, ultrasonic heating, laser heating, magnetic heating, and/or microwave heating.

In some examples, the heated liquid 208 may be used as the treatment liquid (e.g., treatment liquid 154), and thus the fluid reservoir 207 may be in fluid communication with a liquid delivery channel (e.g., liquid delivery channel 162) of the flexible elongate device 202. In some examples, the heated liquid 208 may also be used as a preheat fluid to be flowed into the insulation chamber 160 and evacuated prior to flowing the treatment liquid through the liquid delivery channel 162. In some examples, a preheat fluid may be contained in a reservoir separated from the reservoir containing the heated treatment liquid. In some examples, the temperature control device 210 may heat the treatment liquid to a temperature of less than a vaporization temperature for the treatment liquid. The liquid 208 may be, for example, water, saline, gel, glycerin, solution, or oil that maintains a liquid state at temperatures approaching 100 degrees Celsius. Depending on the components of the liquid, it may be heated to a temperature greater than 100 degrees Celsius while maintaining a liquid state. Glycerin and oil-based liquids may, for example, have boiling points greater than 100 degrees Celsius and thus may be used at temperatures higher than 100 degrees Celsius. In some examples, the liquid may be heated to a temperature between approximately 50 and 200 degrees Celsius. The liquid 208 may include any of the liquid materials or additives described in other embodiments.

An optional pressurization system 212 may be coupled to the reservoir 207 to pressurize the liquid 208 and urge the liquid 208 into the flexible elongate device 202. The pressurization system 212 may pressurize the liquid using, for example, a linear actuator, a screw pump, a piston pump, a rotary pump, a diaphragm pump, or a peristaltic pump. In some examples, the reservoir 207 may be a syringe and may be heated to approximately 98° C. by the temperature control device 210. In some examples, the liquid 208 may be pressurized by heating.

In some embodiments, dedicated valves may be used with any or all of the fluid sources or reservoirs in the medical instrument system. In some embodiments, one or more multi-way valves may be used to control the flow of any or all of the fluid sources. In some embodiments, dedicated pumps, valves, or other flow control mechanisms may be used to provide dedicated control of the activation and speed of flow of fluids from each of the fluids in a fluid source or reservoir. In some embodiments, the temperature, flow rate, flow initiation, flow termination, or other control aspects of liquid delivery through the medical instrument system may be controlled by a robot-assisted medical system.

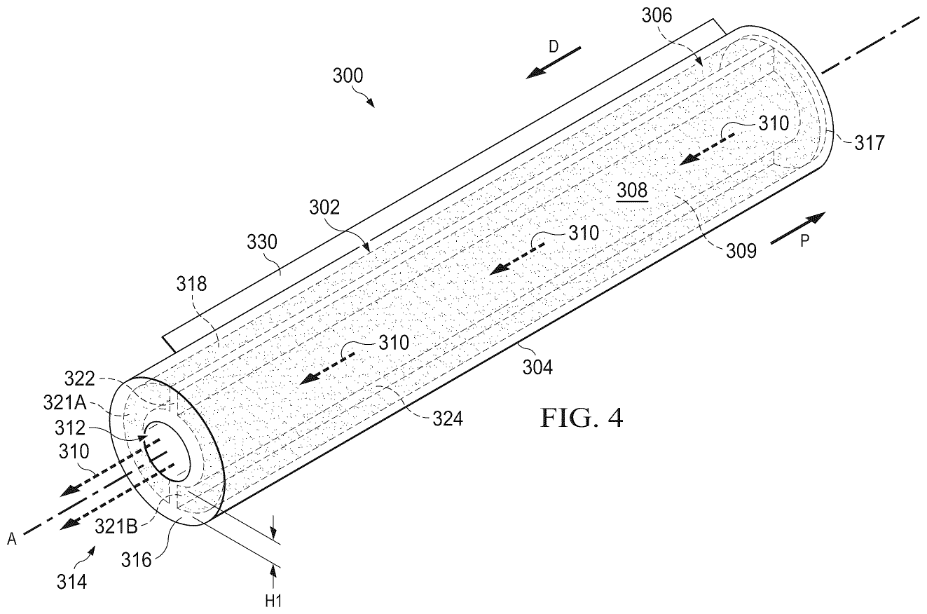

FIG. 4 illustrates a flexible elongate device 300 (e.g., the flexible elongate device 150, 202). In some examples, the flexible elongate device 300 may include an outer wall 304 surrounding a lumen 306. The outer wall 304 may have a diameter sized to extend within the working channel of delivery device (e.g. delivery device 151). A liquid delivery channel 308 extends within the lumen 306. The liquid delivery channel 308 may have a channel wall 309 and may carry a treatment liquid 310 (e.g., the treatment liquid 154) to a distal opening 312 for release to an area 314 distal of the flexible elongate device 300. The diameter of delivery channel 308 may affect the heat transfer to a delivery device (e.g. the delivery device 151). For example, a smaller diameter places the liquid delivery channel 308 further from the working channel of the delivery device and may lower transferred temperatures. The smaller surface area of the smaller liquid delivery channel may, however, cause the liquid delivery channel to become hotter. If the liquid delivery channel has a larger diameter, the liquid delivery channel will be closer to the working channel of the delivery device and may increase transferred temperatures. The increased surface area of the larger liquid delivery channel may, however, cause the delivery channel to be cooler.

An insulation chamber 302 (e.g., insulation chamber 160) may extend along at least a portion of the liquid delivery channel 308. The insulation chamber 302 may be bounded by a distal wall 316 and a proximal wall 317 to resist or prevent inflow or outflow of fluids or other materials. The insulation chamber 302 may contain a static insulator 318 that may be static in that it may not move or circulate into or out of the chamber 302 while the heated treatment liquid 310 flows through the liquid delivery channel 308. In some examples, the insulation chamber 302 may be filled with or contain a static insulator 318 such as air or solid insulation material. The solid insulation material may be a continuous material such as a foam or may include particles such as synthetic down or fiberglass. In other examples, a vacuum May be formed within the insulation chamber 302. The insulation chamber 302 may insulate the liquid delivery channel 308 to maintain or control a temperature of the heated treatment liquid 310 along the length of the flexible elongate device 300. The insulation chamber 302 may reduce, minimize, or eliminate heat transfer between liquid delivery channel 308 and the outer wall 304.

In this example, the insulation chamber 302 includes elongated chamber sections 321A and 321B which have generally C-shaped cross-sections with each chamber section 321A, 321B surrounding approximately half of the liquid delivery channel 308. The chamber sections 321A, 321B may each be bounded or sealed by the wall 316 at the distal end of the flexible elongate device 300. Support structures 322, 324 constrain the axial position of the liquid delivery channel 308 with respect to the wall 304 and lumen 306. In this example, support structure 322 may be an upper septum and the support structure 324 may be a lower septum 324 that separate the adjacent chamber sections 321A, 321B. The septums 322, 324 may be elongated flexible partitions or elongated flexible beams that extend between the outer wall 304 and the channel wall 309 along length of the liquid delivery channel 308. The septums 322, 324 may also extend between the distal wall 316 and the opposite proximal wall 317. The septums 322, 324 may function to maintain the liquid delivery channel 308 in a predetermined axial configuration relative to the outer wall 304 and/or to a central longitudinal axis A. In this example, the septums 322, 324 may have a common height H1 to maintain the liquid delivery channel 308 aligned and co-axial with the central longitudinal axis A. In this example, the septums 322, 324 may be spaced approximately 180 degrees apart about the axis A, but in other examples, the septums may be arranged with different radial spacing. The septums 322, 324 may further contribute to the structural integrity of the flexible elongate device 300 to resist collapse or kinking. In alternative examples, a single septum or more than two septums may be used to form the insulation chamber(s). A septum formed between the outer wall 304 and the channel wall 309 may not provide the same insulative properties as the insulation chamber 302, and thus a septum may provide a conduit for heat transfer between the liquid delivery channel 308 and the outer wall 304. Consequently, in some examples the number of septums and chamber sections may be minimized to reduce localized heating along the length of the outer wall 304.

Optionally, the flexible elongate device 300 may include a fin 330 or longitudinal rib extending along an outer surface of wall 304 to enforce a separation between the wall 304 and an interior wall of a working channel wall of a catheter or other delivery device (e.g. delivery device 151) through which the flexible elongated device 300 extends. The fin 330 may reduce long stretches of direct contact between wall 304 the delivery device.

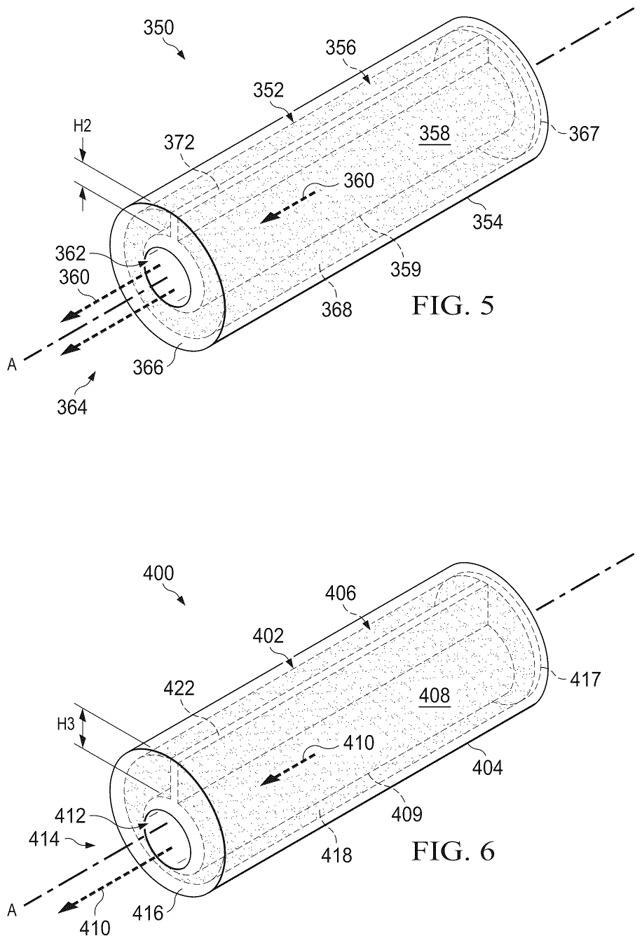

FIG. 5 illustrates a flexible elongate device 350 (e.g., the flexible elongate device 150, 202). In some examples, the flexible elongate device 350 may include an outer wall 354 surrounding a lumen 356. A liquid delivery channel 358 extends within the lumen 356. The liquid delivery channel 358 may have a channel wall 359 and may carry a treatment liquid 360 (e.g., the treatment liquid 154) to a distal opening 362 for release to an area 364 distal of the flexible elongate device 350. An insulation chamber 352 (e.g., insulation chamber 160) may extend along at least a portion of the liquid delivery channel 358. The insulation chamber 352 may be bounded by a distal wall 366 and a proximal wall 367 to resist or prevent inflow or outflow of fluids or other materials. The insulation chamber 352 may contain a static insulator 368 that may be static in that it may not move or circulate into or out of the chamber 352 while the heated treatment liquid 360 flows through the liquid delivery channel 358. In some examples, the insulation chamber 352 may be filled with or contain a static insulator 368 such as air or solid insulation material. The solid insulation material may be a continuous material such as a foam or may include particles such as synthetic down or fiberglass. In other examples, a vacuum may be formed within the insulation chamber 352 The insulation chamber 352 may insulate the liquid delivery channel 358 to maintain or control a temperature of the heated treatment liquid 360 along the length of the flexible elongate device 300. The insulation chamber 352 may reduce, minimize, or eliminate heat transfer between liquid delivery channel 358 and the outer wall 354.

In this example, the insulation chamber 352 includes a single elongated chamber extending around the liquid delivery channel 358. The insulation chamber 352 may be bounded by the wall 366 at the distal end of the flexible elongate device 350. In this example, a support structure 372 may be a septum extending between the outer wall 354 and the channel wall 359 along length of the liquid delivery channel 358. The septum 372 may also extend between the distal wall 366 and the opposite proximal wall 367. The septum 372 may further function to maintain the liquid delivery channel 358 in a predetermined axial configuration relative to the outer wall 354 and/or to a central longitudinal axis A. In this example, the septum 372 may have a height H2 to maintain the liquid delivery channel 358 aligned and co-axial with the central longitudinal axis A. The septum 372 may further contribute to the structural integrity of the flexible elongate device 350 to resist collapse or kinking. As compared to examples in which multiple elongated septums are used, the single elongated septum 372 may localize heating in a single radial direction and generally linearly along the length of the outer wall 354.

FIG. 6 illustrates a flexible elongate device 400 (e.g., the flexible elongate device 150, 202). In some examples, the flexible elongate device 400 may include an outer wall 404 surrounding a lumen 406. A liquid delivery channel 408 extends within the lumen 406. The liquid delivery channel 408 may have a channel wall 409 and may carry a treatment liquid 410 (e.g., the treatment liquid 154) to a distal opening 412 for release to an area 414 distal of the flexible elongate device 400. An insulation chamber 402 (e.g., insulation chamber 160) may extend along at least a portion of the liquid delivery channel 408. The insulation chamber 402 may be bounded by a distal wall 416 and a proximal wall 417 to resist or prevent inflow or outflow of fluids or other materials. The insulation chamber 402 may contain a static insulator 418 that may be static in that it may not move or circulate into or out of the chamber 402 while the heated treatment liquid 410 flows through the liquid delivery channel 408. In some examples, the insulation chamber 402 may be filled with or contain a static insulator 418 such as air or solid insulation material. The solid insulation material may be a continuous material such as a foam or may include particles such as synthetic down or fiberglass. In other examples, a vacuum may be formed within the insulation chamber 402.

In this example, the insulation chamber 402 includes a single elongated chamber extending around the liquid delivery channel 408. The insulation chamber 402 may be bounded or sealed by the wall 416 at the distal end of the flexible elongate device 400. In this example, a support structure 422 may be a septum extending between the outer wall 404 and the channel wall 409 along the length of the liquid delivery channel 408. The septum 422 may also extend between the distal wall 416 and the opposite proximal wall 417. The septum 422 may further function to maintain the liquid delivery channel 408 in a predetermined axial configuration relative to the outer wall 404 and/or to a central longitudinal axis A. In this example, the septum 422 may have a height H3 to maintain the liquid delivery channel 408 parallel to but spaced away from (e.g. non-coaxial with) the central longitudinal axis A. In this example, the height H3 of the septum 422 may be greater than the height H2 of the septum 372 in FIG. 5, and the extra distance and surface area between the liquid delivery channel 408 may dissipate heat, causing lower localized heating in the single radial direction, along the length of the outer wall 454. However, the greater height H3 locates the liquid delivery channel 408 closer to the opposite side of the outer wall 454, thus increasing localized heating in the opposite radial direction from the septum 422. In some examples, the height H3 may be optimized based on material properties, width of the septum 422, and properties of the outer wall 454 to approximately equalize or thermally balance the temperature concentrations along the outer wall 454 at the septum 422 and opposite the septum.

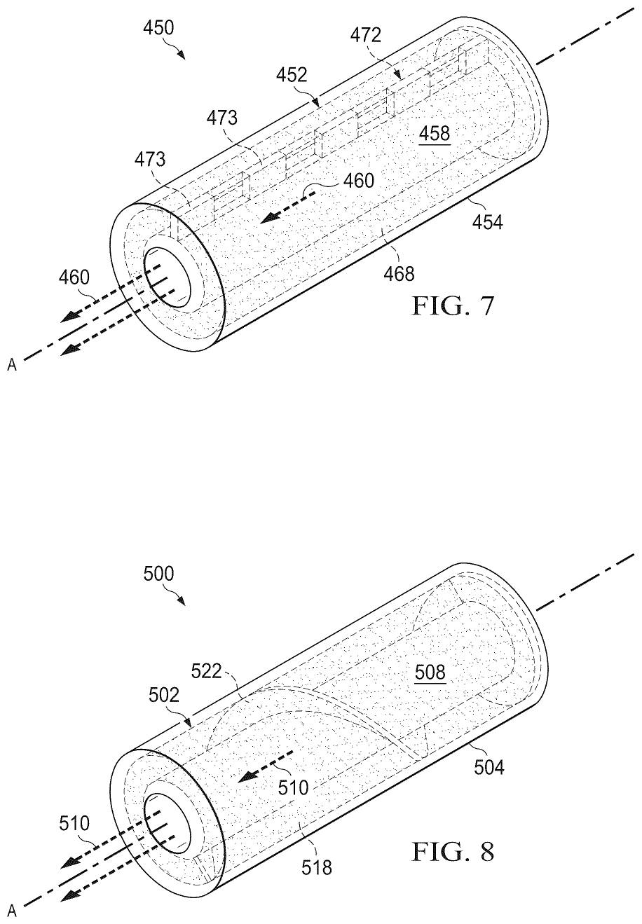

FIG. 7 illustrates a flexible elongate device 450 (e.g., the flexible elongate device 150, 202) that may be substantially similar to flexible elongate device 350, with differences as described. In this example, an insulation chamber 452 extends along at least a portion of a liquid delivery channel 458 that delivers a heated treatment liquid 460. A support structure 472 may be a septum that maintains the liquid delivery channel 458 in a predetermined axial configuration relative to the central longitudinal axis A. In this example, the septum 472 includes a discontinuous series of septal members 473. The insulation chamber 452 may include the space or gaps between the septal members 473 and may be filled with a substance 468 or a vacuum, as previously described, to insulate the liquid delivery channel 458. The septum 472 may function to maintain the liquid delivery channel 458 in a predetermined axial configuration and may contribute to the structural integrity of the flexible elongate device. As compared to the continuous septum in FIG. 5, the gaps between the discontinuous septal members 473 may reduce localized heating along length of the outer wall 454. In some examples, the septal members may have an approximately 1 cm length and may be separated by gaps having an approximately 1 cm length.

FIG. 8 illustrates a flexible elongate device 500 (e.g., the flexible elongate device 150, 202) that may be substantially similar to flexible elongate device 350, with differences as described. In this example, an insulation chamber 502 extends along at least a portion of a liquid delivery channel 508 that delivers a heated treatment liquid 510. A support structure 522 may be a septum that has a spiral shape and maintains the liquid delivery channel 508 in a predetermined axial configuration relative to the central longitudinal axis A. In this example, the spiral septum 522 may extend along a partial or full length of the flexible elongate device 500. The insulation chamber 502 may have a spiral shape that wraps around the liquid delivery channel 508 and may be filled with a substance 518 or a vacuum, as previously described, to insulate the liquid delivery channel 508. The spiral septum 522 may function to maintain the liquid delivery channel 508 in a predetermined axial configuration and may contribute to the structural integrity of the flexible elongate device. As compared to the continuous linear septum in FIG. 5, the spiral septum 522 may distribute heat transfer from the liquid delivery channel 508 in a spiral pattern around the central longitudinal axis A and along the length of the outer wall 504.

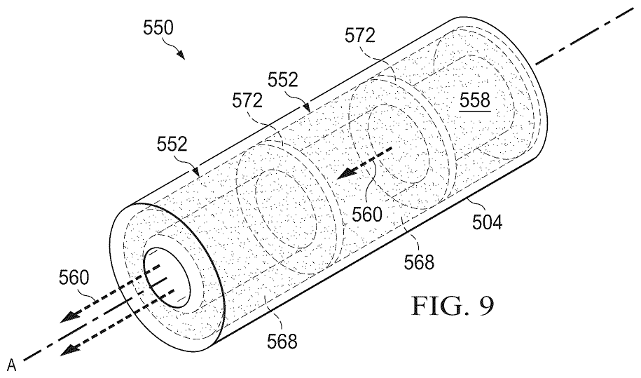

FIG. 9 illustrates a flexible elongate device 550 (e.g., the flexible elongate device 150, 202) that may be substantially similar to flexible elongate device 350, with differences as described. In this example, a series of insulation chambers 552 extend along at least a portion of a liquid delivery channel 558 that delivers a heated treatment liquid 560. A support structure 572 may be a series of septums that ring the liquid delivery channel 558 and maintain the liquid delivery channel 558 in a predetermined axial configuration relative to the central longitudinal axis A. In this example, each of the septums 572 may be a disk-shaped partition that extend generally perpendicular to the central longitudinal axis A, along a partial or full length of the flexible elongate device 550. In other examples, the septums may be skewed relative to the central longitudinal axis A. The insulation chambers 552 may have a tubular shape that surrounds the liquid delivery channel 558 and may be filled with a substance 568 or a vacuum, as previously described, to insulate the liquid delivery channel 558. The series of spaced apart septums 572 may function to maintain the liquid delivery channel 508 in a predetermined axial configuration and may contribute to the structural integrity of the flexible elongate device. As compared to the continuous linear septum in FIG. 5, the series of ringed septums 572 may distribute heat transfer from the liquid delivery channel 558 in a radial pattern around the central longitudinal axis A, at separated, predetermined locations along the length of the outer wall 554.

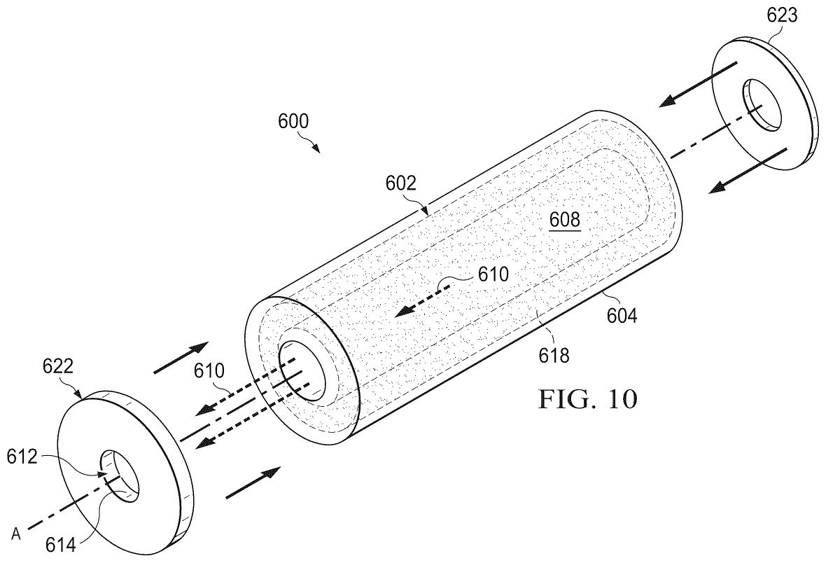

FIG. 10 illustrates a flexible elongate device 600 (e.g., the flexible elongate device 150, 202) that may be substantially similar to flexible elongate device 350, with differences as described. In this example, an insulation chamber 602 extends around and along a liquid delivery channel 608 that delivers a heated treatment liquid 610. In this example, the liquid delivery channel 608 may be maintained in a predetermined axial configuration relative to the central longitudinal axis A by a support structure that may include a distal end cap 622 and an opposite proximal end cap 623. The distal end cap 622 may include a distal aperture or opening 612 aligned with the liquid delivery channel 608 to allow passage of the heated treatment liquid through the distal end cap 622. The distal opening 612 may be surrounded by a cylindrical surface 614 that may extend into the distal end of the liquid delivery channel 608 or extend around the liquid delivery channel to maintain the end of the liquid delivery channel in the predetermined spatial relationship to the axis A and at a predetermined distance from the outer wall 604. The distal end cap 622 may couple to the outer wall 604 of the flexible elongate device 600 and/or to the liquid delivery channel 608 with, for example, an adhesive, threaded, press-fit or other type of connection mechanism. The insulation chambers 602 may have a tubular shape that surrounds the liquid delivery channel 608 and may be filled with a substance 618 or a vacuum, as previously described, to insulate the liquid delivery channel 608. In this example, the omission of the septum may reduce or eliminate concentrated heating along the outer wall 604 at the location of a septum.

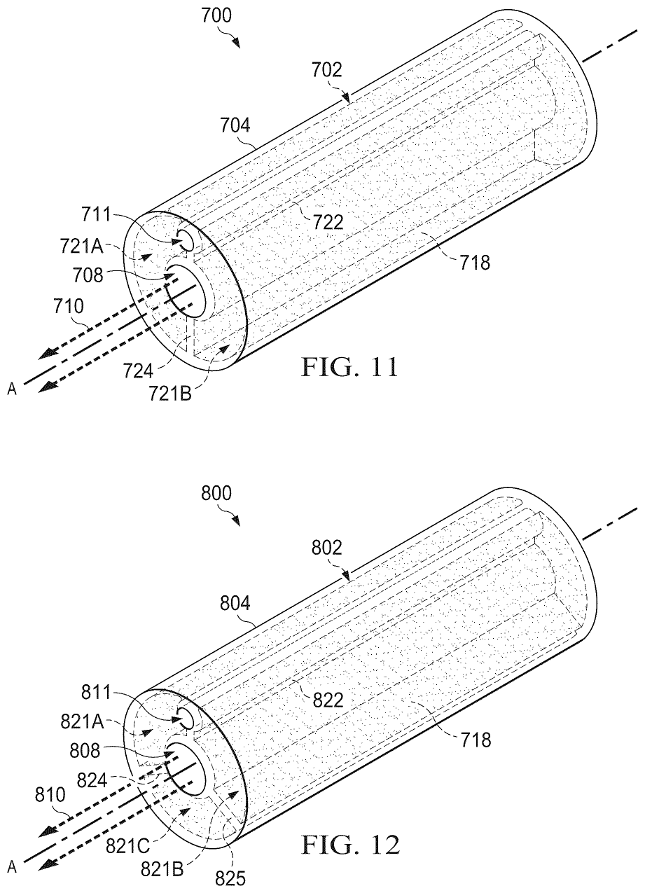

FIG. 11 illustrates a flexible elongate device 700 (e.g., the flexible elongate device 150, 202) that may be substantially similar to flexible elongate device 350, with differences as described. In this example, an insulation chamber 702 extends along at least a portion of a liquid delivery channel 708 that delivers a heated treatment liquid 710. In this example an inflation lumen 711 extends through the flexible elongate device 700 to deliver an inflation medium to an occlusion device (e.g., occlusion device 204). A support structure 722 may be a septum extending between inflation lumen 711 and the liquid delivery channel 708. A support structure 724 may be a septum extending between the liquid delivery channel 708 and an outer wall 704 of the flexible elongate device 700. Supporting the liquid delivery channel 708 with approximately 180 degree spaced septums may improve stiffness to reduce buckling and may reduce the formation of kinks during clinical use. In this example, the insulation chamber 702 includes elongated chamber sections 721A and 721B which have generally C-shaped cross-sections with each chamber section 721A, 721B surrounding approximately half of the liquid delivery channel 708. Support structures 722, 724 constrain the axial position of the liquid delivery channel 708 with respect to the wall 704 and lumen 706. The elongated chamber sections 721A, 721B may be filled with a substance 718 or a vacuum, as previously described, to insulate the liquid delivery channel 708.

FIG. 12 illustrates a flexible elongate device 800 (e.g., the flexible elongate device 150, 202) that may be substantially similar to flexible elongate device 350, with differences as described. In this example, an insulation chamber 802 extends along at least a portion of a liquid delivery channel 708 that delivers a heated treatment liquid 810. In this example an inflation lumen 811 extends through the flexible elongate device 800 to deliver an inflation medium to an occlusion device (e.g., occlusion device 204). A support structure 822 may be a septum extending between inflation lumen 811 and the liquid delivery channel 808. A support structure 824 and a support structure 825 may be septums extending between the liquid delivery channel 808 and an outer wall 804 of the flexible elongate device 800. The septums 822, 824, 825 may be spaced approximated 120 degrees apart about the longitudinal axis A. Supporting the liquid delivery channel 808 with more than two equally spaced septums may improve stiffness to reduce buckling and may reduce the formation of kinks during clinical use. In this example, the insulation chamber 802 includes elongated chamber sections 821A, 821B, 821C with each chamber section surrounding approximately one-third of the liquid delivery channel 808. Support structures 822, 824, 825 constrain the axial position of the liquid delivery channel 808 with respect to the wall 804 and lumen 806. The elongated chamber sections 821A, 821B, 821C may be filled with a substance 818 or a vacuum, as previously described, to insulate the liquid delivery channel 808.

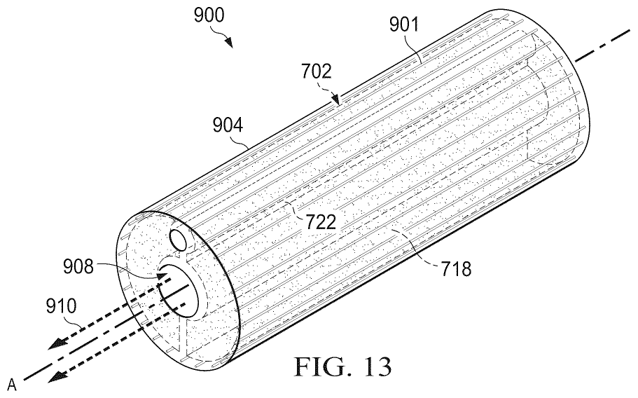

FIG. 13 illustrates a flexible elongate device 900 (e.g., the flexible elongate device 150, 202) that may be substantially similar to flexible elongate device 700, with differences as described. In this example, thermal expansion mitigation members may form a reinforcement system 901 that may extend within an outer wall 904 of the flexible elongate device 900 to mitigate thermal expansion that may be promoted by the heated liquid 910 flowing through a liquid delivery channel 908. In some examples, the thermal expansion mitigation members may include reinforcement fibers that extend entirely or primarily parallel to the longitudinal axis A to prevent elongation of the flexible elongate device while the heated liquid is flowing. In some examples the reinforcement system may be comprised of flexible fibers or a flexible fiber mesh. Flexible fibers may include, for example, aramid reinforcement wires.

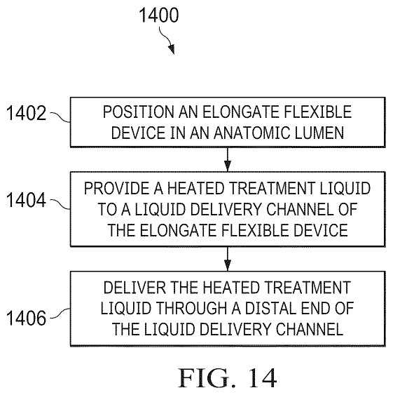

FIG. 14 is a flowchart illustrating a method 1400 for applying a thermal energy treatment to an endoluminal passageway. The method 1400 is illustrated as a set of operations or processes that may be performed in the same or in a different order than the order shown in FIG. 14. One or more of the illustrated processes may be omitted in some embodiments of the method. Additionally, one or more processes that are not expressly illustrated in FIG. 14 may be included before, after, in between, or as part of the illustrated processes. In some embodiments, one or more of the processes of method 1400 may be implemented, at least in part, by a control system executing code stored on non-transitory, tangible, machine-readable media that when run by one or more processors (e.g., the processors of a control system) may cause the one or more processors to perform one or more of the processes.

At a process 1402, a flexible elongate device of a medical instrument system, such as any of the flexible elongate devices previously described, may be positioned in an anatomic passageway (e.g., a passageway 102). Pulmonary blood vessels or vasculature may extend alongside the bronchial passageway 102. A target tissue for treatment with the medical instrument system, which may be, for example a lung tumor, may be located distally of or downstream from the positioned distal end of the flexible elongate device. In some examples, the target tissue may be located throughout a region of the anatomy (e.g., region 113). The positioning of the flexible elongate device may be performed with a robot-assisted endoluminal medical system or may be performed with an endoscope manually by a clinician. In some examples, process 1402 may be optional and the method 1400 may begin after the flexible elongate device is positioned.

At a process 1404, a heated treatment liquid may be provided to a liquid delivery channel of the flexible elongate device. The liquid delivery channel may be insulated by an insulation chamber. For example, an insulation chamber may be filled with a static insulator substance (e.g., the static insulator material 169, 318, 368, 418, 468, 518, 568, 618) such as air or another insulation material such as foam, synthetic down, fiberglass, or other synthetic material. In other examples, a static insulator may be a vacuum formed within the insulation chamber. The insulation chamber may insulate the liquid delivery channel to maintain or control a temperature of the heated treatment liquid along the length of the flexible elongate device. The insulation chamber may reduce, minimize, or eliminate heat transfer between liquid delivery channel and the outer wall of the flexible elongate device. The insulation chamber may be a static insulation chamber in that material may not move or circulate into or out of the chamber while the heated treatment liquid flows through the liquid delivery channel. Optionally, one or more insulation chambers adjacent to a liquid delivery channel may be preheated. For example, heated air may be pumped into an insulation chamber (e.g., insulation chamber 160, 302, 352, 402, 452, 502, 552, 602) before the heated treatment liquid is flowed through the liquid delivery channel. The heated air may heat the outer wall of the liquid delivery channel. The heated air may be sealed in the insulation chamber or may be vacated prior to scaling a vacuum or an insulation material in the insulation chamber 160. In other examples, a heated liquid may be pumped into the insulation chamber to heat the wall of the liquid delivery channel. The heated liquid may be evacuated, and the insulation chamber dried before a vacuum or an insulating substance is scaled in the insulation chamber.

Prior to entering the liquid delivery channel, the heated treatment liquid may be heated and contained, for example, in a reservoir (e.g. reservoir 207) at a temperature of between approximately 95° C. and 99° C. The heated treatment liquid may be injected, pumped, or otherwise conveyed into the liquid delivery channel. In some embodiments, the temperature of the heated treatment liquid may be maintained at or near a target delivery temperature of between approximately 95° C. and 99° C. by the insulation provided by the insulation chamber(s) while in transit along the liquid delivery channel. Without the insulation chamber(s), the temperature of the treatment liquid could drop during transit through the flexible elongate device to an unacceptable temperature for treatment in the anatomic passageway, with longer flexible elongate devices experiencing greater drops in temperature.

At a process 1406, a heated treatment liquid is delivered through a distal end of the liquid delivery channel. The heated treatment liquid may be dispensed from the liquid delivery channel into the anatomic lumen. In some embodiments, the released heated treatment liquid may directly contact the walls of the anatomic lumen causing ablation at and/or near the target tissue. In other embodiments, the released heated treatment liquid may flow into an expandable device such as a silicone balloon that may contain the heated treatment liquid but allow the transfer of heat to the adjacent tissue to ablate the tissue. In such examples, an occlusion balloon may be omitted. After the ablation with the heated balloon, the heated treatment liquid may be evacuated through the liquid delivery channel and may, in some examples, return to a fluid reservoir. Whether ablated by direct contact with the treatment liquid or by a balloon filled with the treatment liquid, the depth of ablation and therefore the anatomical structures (e.g., bronchial passageway, bronchial artery, pulmonary artery, etc.) occluded by the ablation may be controlled, for example, based on the amount of liquid released from the flexible elongate device and the temperature of the heated treatment liquid. Ablation may induce cellular and structural changes in the epithelium that in some cases may extend to the sub-epithelium. The ablation may cause tissue reduction, including destruction of goblet cells and cilia in lung tissue. In some embodiments, the cellular matrix may be preserved to allow for later regrowth of healthy cells. In some examples, the tissue reaction may occur entirely during the application of the heated treatment liquid, and in other examples, the tissue damage may develop over a period of time as the anatomy responds to the injury caused by the heat. A proximal flow of the heated treatment liquid in the anatomic lumen may be restricted by an occlusion device (e.g., an occlusion balloon), thus urging the dispensed treatment liquid into an area of the anatomic passageway distal of the flexible elongate device.

In some embodiments, the flexible elongate device may be moved (e.g., retracted) during the delivery of the heated treatment liquid. In some embodiments, the movement may be performed manually. In some embodiments, the treatment device may be coupled to a manipulator of a robot-assisted medical system (e.g., a system 800) and movement of the treatment device from a first location to a second location may be performed by actuation of a manipulator. In some embodiments, an occlusion device can remain inflated during retraction or might need to be deflated slightly during retraction. The amount of deflation may, for example, be based on sensed pressure, be a predetermined delta from the inflated state, or be determined based on visual feedback (e.g., user determined or by image recognition).

If the heated treatment liquid raises the temperature on the outside surface of the outer wall of the flexible elongate device for an extended period of time, the flexible elongate device may damage the adjacent anatomic tissue or delivery device. Thus, this treatment method may maintain the treatment temperature of the treatment liquid while maintaining an external temperature along the flexible elongate device than minimizes thermal risk to the adjacent tissue or equipment. In some examples, an outside temperature of the outer wall of the flexible elongate device may be maintained at a pre-determined safety temperature of, for example, 70° C. Temperature sensors may be included within or along the outer wall of the flexible elongate device to measure temperature, and the duration of flow may be altered based on the sensed temperature, in a closed loop manner. In some examples, the flow rate, flow duration, and/or fluid temperature may be altered based on temperature of the flexible elongate device wall. The temperature of the treatment fluid may be monitored (e.g., with a temperature sensor within the delivery fluid lumen). In some embodiments the temperature of the treatment fluid may be monitored along different lengths of the delivery fluid lumen, such as at a proximal location, a distal location immediately before fluid exit from the delivery channel, or multiple points in between to determine change in temperature as fluid is delivered down the length of the flexible elongate device.

As described above, an occlusion device (e.g. occlusion device 204) may be inflated in an anatomic passageway, proximal of the released heated fluid, to prevent proximal flow of the fluid and encourage movement of the fluid to the desired treatment area. Depending on the characteristics of the anatomy where the treatment is needed, various techniques may be employed to minimize leakage by promoting a seal between the occlusion device and surrounding airway. The various techniques may also or alternatively place the distal opening of a liquid delivery channel at a sufficient distance from a distal bifurcation in the anatomic passageway to ensure that heated fluid may flow into both distal passages. The occlusion device may flex to conform to the size and shape of the passageway or the opening to a passageway, even in tortuous and convoluted geometries, to maintain a full circumferential seal. In some geometries, the flexible elongate device may be non-concentric to the flexed occlusion device to allow for improved apposition to the passageway wall. The use of the occlusion device may allow for treatment at segmental, subsegmetal, or even sub-subsegmental generations of passageways. In some examples, radial pressure on the airway provided by the occlusion device may at least partially occlude flow of blood through arteries that extend just beneath the surface of the passageway wall. Slowing or stopping blood flow may improve the conditions for ablation by reducing perfusion of the distal passageways and potentially leading to improved lung volume reduction.

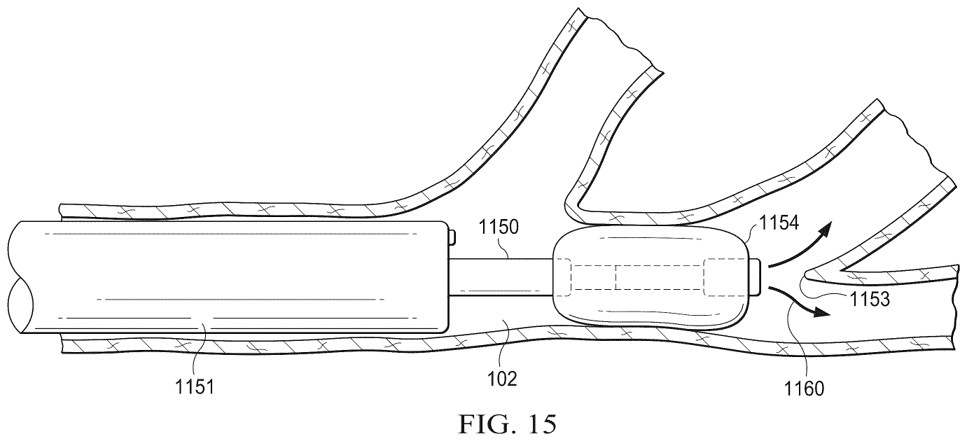

FIG. 15 illustrates an anatomic passageway 102 in which a flexible elongate device 1150 (e.g., the flexible elongate device 150, 202) is extended from a flexible elongate delivery device 1151 (e.g., the flexible elongate delivery device 151). The distal end of the flexible elongate device 1150 may located a sufficient distance from a bifurcation 1153 to allow a heated fluid 1160 to flow into both distal passageways. An occlusion device 1154 (e.g., the occlusion device 204) may be inflated around the flexible elongate device 1150 to occlude the anatomic passageway 102 and prevent the proximal flow of the heated fluid 1160 that exits the distal end portion of the flexible elongate device 1150. FIG. 15 illustrates a luminal technique (e.g., within the lumen of passageway 102) for placement of the occlusion device 1154, which may be particularly suitable for relatively long anatomic passages. In this example, the occlusion device 1154 may be inflated within the passageway 102, creating a seal between the occlusion device 1154 and the passageway wall at approximately the widest or midline portion of the occlusion device. In some examples, the occlusion device may be approximately 20% overinflated relative to the diameter of the passageway 102.

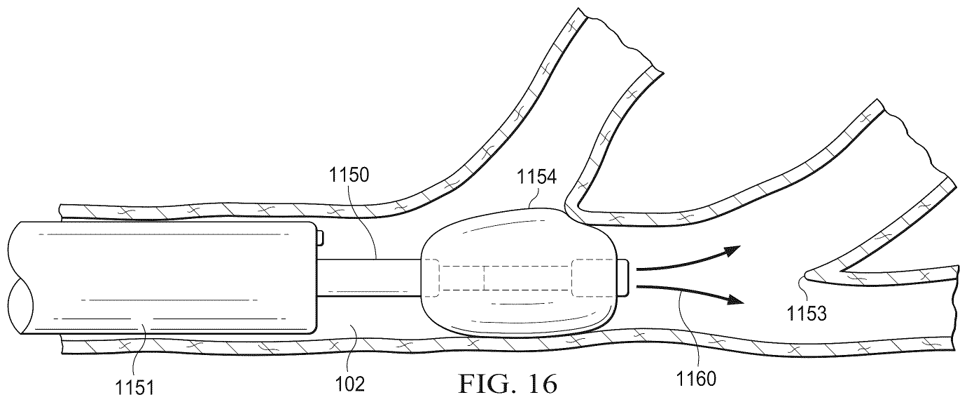

FIG. 16 illustrates an ostial abutment technique (e.g., at an opening to the lumen of passage way 102) for placement of the occlusion device 1154, which may be particularly suitable for relatively short anatomic passages. In this example, the occlusion device 1154 may be inflated generally outside the passageway 102 (e.g., in a passageway one generation proximal of the passageway 102), and a distal (e.g., forward) force may be applied to the flexible elongate device 1150 to create a seal between the occlusion device 1154 and the opening of the passageway 102 at a distal surface of the occlusion device. In some examples, the occlusion device may be approximately 30-50% overinflated relative to the diameter of the passageway 102.

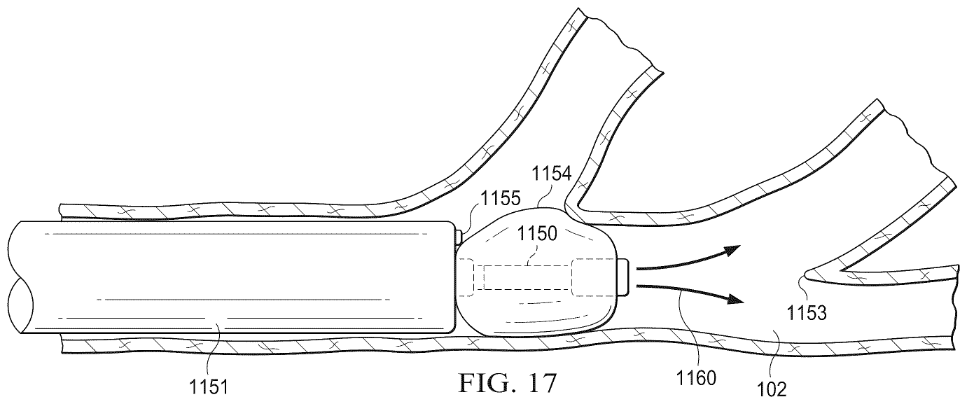

FIG. 17 illustrates an alternative ostial abutment technique (e.g., at an opening to the lumen of passageway 102) for placement of the occlusion device 1154, which may be particularly suitable for relatively short anatomic passages. In this example, the occlusion device 1154 may be inflated generally outside the passageway 102 (e.g., in a passageway one generation proximal of the passageway 102), and a distal (e.g., forward) force may be applied to the flexible elongate device to create a seal between the occlusion device 1154 and the opening of the passageway 102 at a distal surface of the occlusion device. In some examples, the occlusion device may be approximately 30-50% overinflated relative to the diameter of the passageway 102. In this example, the flexible elongate delivery device 1151 may be pushed against or moved into abutment with a proximal surface of the occlusion device 1154 to help hold the occlusion device 1154 in place and to create a “fishbowl” view for an imaging system 1155 of the delivery device 1151. Thus, the imaging system 1155 may allow an operator to better visualize the bifurcation 1153 and select a location for releasing the fluid 1160 that allows for flow into both distal passageways.

In some examples, the systems and methods disclosed herein may be used in a medical procedure performed with a robot-assisted medical system as described in further detail below. FIG. 12 is a simplified diagram of a medical system 800 according to some embodiments. The medical system 800 may be suitable for use in, therapeutic procedures such as ablation or electroporation. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose robot-assisted medical systems.

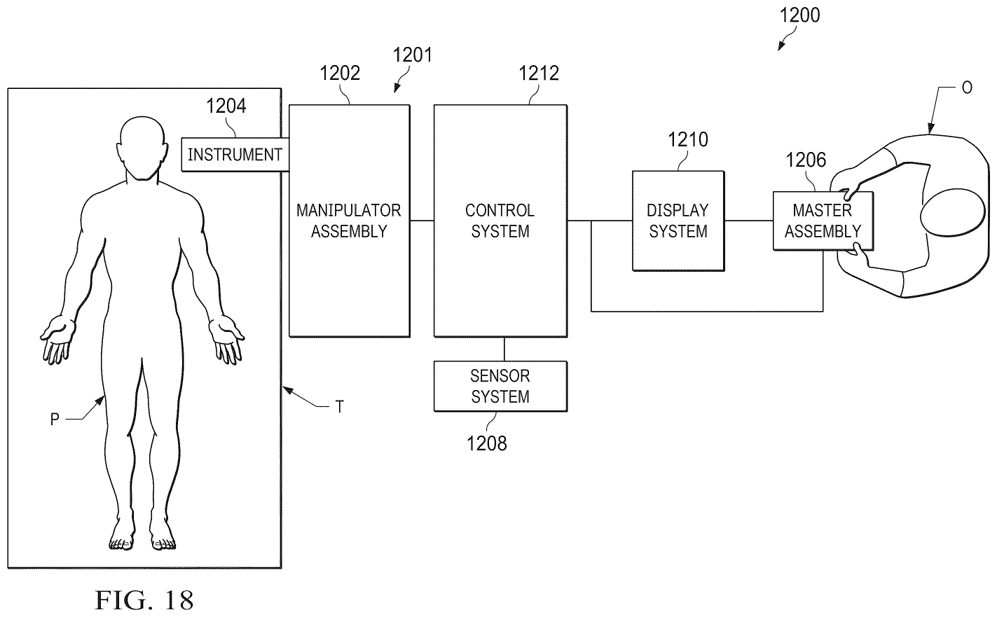

As shown in FIG. 18, a medical system 1200 may include a manipulator assembly 1202 that controls the operation of a medical instrument 1204 in performing various procedures on a patient P. The medical instrument 1204 may be, for example, the medical instrument system 100. In some examples, a flexible elongate device of the medical instrument 1204 may be, for example, the flexible elongate device 150, 202, 300, 350, 400, 450, 500, 550, or 600. In other examples, the medical instrument 1204 may include a robotically-assisted flexible elongate device (e.g. flexible elongate delivery device 151) through which extends a medical tool which may be, for example, the flexible elongate device 150, 202, 300, 350, 400, 450, 500, 550, or 600. Medical instrument 1204 may extend into an internal site within the body of patient P via an opening in the body of patient P. The manipulator assembly 1202 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. The manipulator assembly 1202 may be mounted to and/or positioned near a patient table T. A master assembly 1206 allows an operator O (e.g., a surgeon, a clinician, a physician, or other user) to control the manipulator assembly 1202. In some examples, the master assembly 1206 allows the operator O to view the procedural site or other graphical or informational displays. In some examples, the manipulator assembly 1202 may be excluded from the medical system 1200 and the instrument 1204 may be controlled directly by the operator O. In some examples, the manipulator assembly 1202 may be manually controlled by the operator O. Direct operator control may include various handles and operator interfaces for hand-held operation of the instrument 1204.

The master assembly 1206 may be located at a surgeon's console which is in proximity to (e.g., in the same room as) a patient table T on which patient P is located, such as at the side of the patient table T. In some examples, the master assembly 1206 is remote from the patient table T, such as in in a different room or a different building from the patient table T. The master assembly 1206 may include one or more control devices for controlling the manipulator assembly 1202. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 1202 supports the medical instrument 1204 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 1212). The manipulator assembly 1202 may include a plurality of actuators (e.g., motors) that drive inputs on the medical instrument 1204 in response to commands, such as from the control system 1212. The actuators may include drive systems that move the medical instrument 1204 in various ways when coupled to the medical instrument 1204. For example, one or more actuators may advance medical instrument 1204 into a naturally or surgically created anatomic orifice. Actuators may control articulation of the medical instrument 1204, such as by moving the distal end (or any other portion) of medical instrument 1204 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 1204, such as for grasping tissue in the jaws of a biopsy device and/or the like or may be used to move or otherwise control tools (e.g., imaging tools, ablation tools, biopsy tools, electroporation tools, etc.) that are inserted within the medical instrument 1204.

The medical system 1200 may include a sensor system 1208 with one or more sub-systems for receiving information about the manipulator assembly 1202 and/or the medical instrument 1204. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 1204; a visualization system for capturing images, such as from the distal end of medical instrument 1204 or from some other location; and/or actuator position sensors such as resolvers, encoders, potentiometers, and the like that describe the rotation and/or orientation of the actuators controlling the medical instrument 1204.

The medical system 1200 may include a display system 1210 for displaying an image or representation of the procedural site and the medical instrument 1204. Display system 1210 and master assembly 1206 may be oriented so physician O can control medical instrument 1204 and master assembly 1206 with the perception of telepresence.

In some embodiments, the medical instrument 1204 may include a visualization system 1209, which may include an image capture assembly that records a concurrent or real-time image of a procedural site and provides the image to the operator O through one or more displays of display system 1210. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 1204. Additionally or alternatively, a separate endoscope, attached to a separate manipulator assembly, may be used with medical instrument 1204 to image the procedural site. The visualization system may be implemented as hardware, firmware, software or a combination thereof which interact with or are otherwise executed by one or more computer processors, such as of the control system 1212.

Display system 1210 may also display an image of the procedural site and medical instruments, which may be captured by the visualization system. In some examples, the medical system 1200 provides a perception of telepresence to the operator O. For example, images captured by an imaging device at a distal portion of the medical instrument 1204 may be presented by the display system 1210 to provide the perception of being at the distal portion of the medical instrument 1204 to the operator O. The input to the master assembly 1206 provided by the operator O may move the distal portion of the medical instrument 1204 in a manner that corresponds with the nature of the input (e.g., distal tip turns right when a trackball is rolled to the right) and results in corresponding change to the perspective of the images captured by the imaging device at the distal portion of the medical instrument 1204. As such, the perception of telepresence for the operator O is maintained as the medical instrument 1204 is moved using the master assembly 1206. The operator O can manipulate the medical instrument 1204 and hand controls of the master assembly 1206 as if viewing the workspace in substantially true presence, simulating the experience of an operator that is physically manipulating the medical instrument 1204 from within the patient anatomy.

In some examples, the display system 1210 may present virtual images of a procedural site that are created using image data recorded pre-operatively (e.g., prior to the procedure performed by the medical instrument system 100) or intra-operatively (e.g., concurrent with the procedure performed by the medical instrument system 100), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 1210 may display a virtual image that is generated based on tracking the location of medical instrument 1204. For example, the tracked location of the medical instrument 1204 may be registered (e.g., dynamically referenced) with the model generated using the pre-operative or intra-operative images, with different portions of the model correspond with different locations of the patient anatomy. As the medical instrument 1204 moves through the patient anatomy, the registration is used to determine portions of the model corresponding with the location and/or perspective of the medical instrument 1204 and virtual images are generated using the determined portions of the model. This may be done to present the operator O with virtual images of the internal procedural site from viewpoints of medical instrument 1204 that correspond with the tracked locations of the medical instrument 1204.

The medical system 1200 may also include the control system 1212, which may include processing circuitry that implements the some or all of the methods or functionality discussed herein. The control system 1212 may include at least one memory 1216 and at least one processor 1214 for controlling the operations of the manipulator assembly 1202, the medical instrument 1204, the master assembly 1206, the sensor system 1208, and/or the display system 1210. Control system 1212 may include programmed instructions (e.g., a non-transitory machine-readable medium storing the instructions) that when executed by the at least one processor, configures the one or more processors to implement some or all of the methods or functionality discussed herein. For example, the programmed instructions may implement some or all of the processes described in accordance with aspects disclosed herein, including, for example, expanding an expandable device, regulating a temperature of the heating system, regulating valves to control fluid delivery, controlling fluid flow rate, controlling insertion and retraction of the treatment instrument, controlling actuation of a distal end of the treatment instrument, receiving sensor information, altering signals based on the sensor information, and/or selecting a treatment location. While the control system 1212 is shown as a single block in FIG. 18, the control system 1212 may include two or more separate data processing circuits with one portion of the processing being performed at the manipulator assembly 1202, another portion of the processing being performed at the master assembly 1206, and/or the like. In some examples, the control system 1212 may include other types of processing circuitry, such as application-specific integrated circuits (ASICs) and/or field-programmable gate array (FPGAs). The control system 1212 may be implemented using hardware, firmware, software, or a combination thereof.

In some examples, the control system 1212 may receive feedback from the medical instrument 1204, such as force and/or torque feedback. Responsive to the feedback, the control system 1212 may transmit signals to the master assembly 1206. In some examples, the control system 1212 may transmit signals instructing one or more actuators of the manipulator assembly 1202 to move the medical instrument 1204. In some examples, the control system 1212 may transmit informational displays regarding the feedback to the display system 1210 for presentation or perform other types of actions based on the feedback.

The control system 1212 may include a virtual visualization system to provide navigation assistance to operator O when controlling the medical instrument 1204 during an image-guided medical procedure. Virtual navigation using the virtual visualization system may be based upon an acquired pre-operative or intra-operative dataset of anatomic passageways of the patient P. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 1212 may use a pre-operative image to locate the target tissue (using vision imaging techniques and/or by receiving user input) and create a pre-operative plan, including an optimal first location for performing bronchial passageway and vasculature occlusion. The pre-operative plan may include, for example, a planned size to expand the expandable device, a treatment duration, a treatment temperature, and/or multiple deployment locations.

Medical system 1200 may further include operations and support systems (not shown) such as illumination systems, steering control systems, irrigation systems, and/or suction systems. In some embodiments, the medical system 1200 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 1212) or may be implemented, at least in part, in the form of executable code stored on non-transitory, tangible, machine-readable media that when run by one or more processors (e.g., the processors 1214 of control system 1212) may cause the one or more processors to perform one or more of the processes.

One or more components of the embodiments discussed in this disclosure, such as control system 1212, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom-e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

While certain exemplary examples of the invention have been described and shown in the accompanying drawings, it is to be understood that such examples are merely illustrative of and not restrictive on the broad invention, and that the examples of the invention not be limited to the specific constructions and arrangements shown and described, since various other alternatives, modifications, and equivalents will be appreciated by those with ordinary skill in the art.

1. A flexible elongate device comprising:
a wall defining a lumen;
a liquid delivery channel extending within the lumen, the liquid delivery channel configured to convey a treatment liquid;
an insulation chamber extending along the liquid delivery channel within the lumen, the insulation chamber containing a static insulator; and
a support structure that constrains an axial position of the liquid delivery channel with respect to the lumen.

2. The flexible elongate device of claim 1, wherein the static insulator includes a vacuum.

3. The flexible elongate device of claim 1, wherein the static insulator includes a fluid and wherein the insulation chamber is sealed to prevent inflow or outflow of the fluid.

4. The flexible elongate device of claim 3, wherein the fluid includes a gas.

5. (canceled)

6. The flexible elongate device of claim 1, wherein the static insulator includes a solid insulation material.

7. (canceled)

8. The flexible elongate device of claim 1, wherein the support structure includes a septum extending within the lumen between the wall and the liquid delivery channel.

9. The flexible elongate device of claim 8, wherein the septum maintains the liquid delivery channel approximately co-axial with a longitudinal axis of the lumen.

10. The flexible elongate device of claim 8, wherein the septum maintains the liquid delivery channel along an axis that is non-coaxial with a longitudinal axis of the lumen.

11. The flexible elongate device of claim 8, wherein the septum is a first septum, and the flexible elongate device further comprises a second septum extending within the lumen approximately 180 degrees from the first septum.

12. The flexible elongate device of claim 11, wherein the first septum is longer than the second septum.

13. The flexible elongate device of claim 11, wherein the septum includes a discontinuous series of septal members.

14. The flexible elongate device of claim 11, wherein the septum has a spiral shape.

15. The flexible elongate device of claim 11, wherein the septum includes a ring extending perpendicular to a longitudinal axis of the lumen.

16. The flexible elongate device of claim 8, wherein the septum is a first septum, and the flexible elongate device further comprises a second septum and a third septum, each extending within the lumen approximately 120 degrees from the first septum.

17. The flexible elongate device of claim 1, wherein the support structure includes an end cap coupled to a distal end portion of the flexible elongate device to maintain the liquid delivery channel at a predetermined distance from the wall.

18. (canceled)

19. The flexible elongate device of claim 17, wherein the insulation chamber includes a space extending between the wall and the liquid delivery channel.

20. (canceled)

21. The flexible elongate device of claim 17, wherein the end cap includes an aperture configured to allow passage of the treatment liquid from the liquid delivery channel.

22. The flexible elongate device of claim 1, further comprising a rib or an inflatable occlusion device extending along at least a portion of an outer surface of the wall.

23. (canceled)

24. The flexible elongate device of claim 1, wherein the wall includes thermal expansion mitigation members.

25. The flexible elongate device of claim 1, wherein an inflation lumen extends within the lumen.

26-47. (canceled)
